(19) United States
(12) Patent Application Publication
(10) Pub. No.: US 2025/0002465 A1
(43) Pub. Date: Jan. 2, 2025

(54) TRIMERIC COPPER CATALYSTS, INTERMEDIATES FOR MAKING SUCH CATALYSTS AND METHOD FOR PREPARING A CYCLOADDITION COMPOUND

(71) Applicant: University of Kentucky Research Foundation, Lexington, KY (US)

(72) Inventors: Aron Huckaba, Lexington, KY (US); Alexander Olivelli, Lexington, KY (US)

(21) Appl. No.: 18/750,348

(22) Filed: Jun. 21, 2024

Related U.S. Application Data
(60) Provisional application No. 63/522,676, filed on Jun. 22, 2023.

(51) Int. Cl. C07D 249/04; B01J 31/18; B01J 31/22

(52) U.S. Cl. CPC C07D 249/04; B01J 31/1815; B01J 31/2295; B01J 2231/324; B01J 2531/004; B01J 2531/0205; B01J 2531/16

(57) ABSTRACT

Novel compounds including trimeric copper catalysts and intermediates for making such compounds are presented along with methods for preparing and using those compounds.

### RELATED APPLICATION

This application claims priority to U.S. Provisional Patent Application Ser. No. 63/522,676, filed on Jun. 22, 2023, the full disclosure of which is incorporated herein by reference.

### TECHNICAL FIELD

This document relates to new compounds useful as intermediates for the production of copper-catalyzed azide-alkyne cycloaddition (CuAAC) reaction catalysts, new CuAAC reaction catalysts as well as to methods for their production and use.

### BACKGROUND

Cu-catalyzed azide-alkyne cycloaddition (CuAAC) allows for the rapid assembly of densely functional molecular fragments, even in biologically relevant media and in/on live cells. The first approaches by Sharpless used CuSO₄ and sodium ascorbate or Cu⁰ while Finn reported tri(azolyl)amine Cu complexes and their excellent catalytic properties. One of the few drawbacks of this reaction is the propensity for the sodium ascorbate and catalytic Cu ion to contribute to the generation of reactive oxygen species (ROS). These reactive oxygen species oxidatively stress cells and can oxidize essential biological molecules.

Exclusion of oxygen during the reaction is one method that's been employed to address the generated ROS, as has an excess of amine ligand. Strain-promoted versions (spAAC) of the reaction offer a way around the problematic Cu ions, but they can be slower reactions. The gold standard CuAAC catalyst, Cu(II) tris(hydroxypropyltriazolyl)methylamine (THPTA), has been used in many and is available commercially as a kit. One way to improve on the current catalyst system being used is to find a catalyst that maintains high activity at very low catalyst loadings (<1 mol %) at very low concentrations (<1 mM) in biologically relevant media like THPTA, without generating ROS.

This document relates to the synthesis and characterization of novel Cu(I) based catalysts for the CuAAC reaction. The catalyst structures are trinuclear dimer based on benzimidazole chelating groups attached to a cis, cis,-1,3,5-cyclohexane scaffold. The dimer complex is stable in air, and is not oxidized by oxidants like H₂O₂.

### SUMMARY

In accordance with the purposes and benefits set forth herein, a compound is provided having the chemical formula:

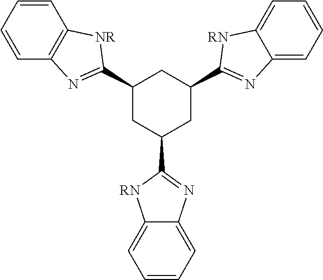

where R=—H, —C₄H₉ or —CH₂—CH₂—O—CH₂—CH₂—O—CH₃.

In accordance with yet another aspect, a compound is provided having the chemical formula:

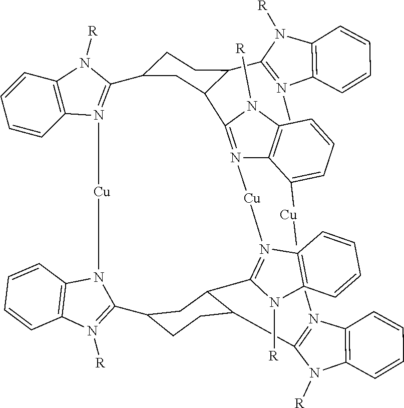

where R=—C₄H₉ or —CH₂—CH₂—O—CH₂—CH₂—O—CH₃.

In accordance with yet another aspect, a method of producing a CuAAC reaction catalyst, comprises, consists of or consists essentially of:
using as a reaction intermediate, a compound selected from a group consisting of:

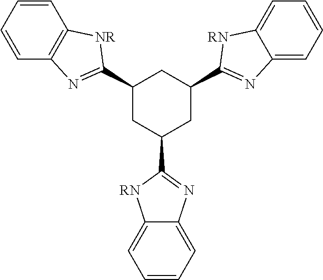

where R=—H, —C₄H₉ or —CH₂—CH₂—O—CH₂—CH₂—O—CH₃; or

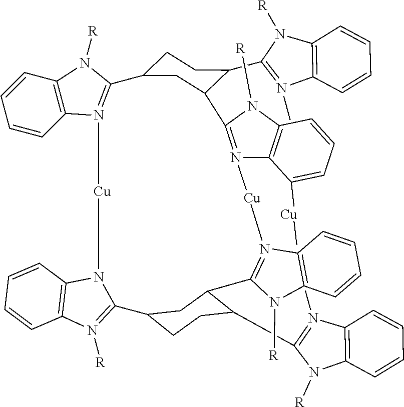

where R=—C₄H₉ or —CH₂—CH₂—O—CH₂—CH₂—O—CH₃.

In accordance with still another aspect, a method of producing a CuAAC reaction catalyst, comprises, consists of or consists essentially of reacting a cis-cis-1,3,5-cyclohexanetribenzimidazole-2-yl-based compound with tetrakis(acetonitrile) copper(I) hexafluorophosphate to form the CuACC reaction catalyst.

In accordance with yet another aspect, a method for preparing a cycloaddition compound from a reaction catalyzed by a heterogeneous copper catalyst, comprises, consists of or consists essentially of: (a) mixing an alkyne component and a azide component in an appropriate solvent to form a solution and (b) contacting the solution with a CuAAC reaction copper(I) catalyst derived from a cis-cis-1,3,5-cyclohexanetribenzimidazole-2-yl-based intermediate compound wherein, upon contact with the CuAAC reaction copper(I) catalyst, the alkyne component and the azide component react to form the cycloaddition compound.

The method may also include the step of completing the contacting of the solution in the absence of a reducing agent. Still more particularly, the method may include completing the contacting of the solution in the absence of sodium ascorbate.

In the following description, there are shown and described several different novel compounds and methods for making and using the same. As it should be realized, the compounds and methods are capable of other, different embodiments and their several details are capable of modification in various, obvious aspects all without departing from the compounds and methods as set forth and described in the following claims. Accordingly, the drawings and descriptions should be regarded as illustrative in nature and not as restrictive.

### BRIEF DESCRIPTION OF THE DRAWING FIGURES

FIG. 1 illustrates the cyclic voltammogram of 3 (dashed line) and 7 (full line) under N₂ in dry and degassed dichloromethane. Voltammogram of 3 trace taken in 0.1 M NBu₄PF₆ supporting electrolyte using Pt pseudo-reference, Pt counter, and glassy carbon working electrodes at scan rate of 100 mV/s.

FIG. 2 illustrates Left: UV-vis absorbance spectra of 3+(CH₃CN)₄CuPF₆ in H₂O after 18 hours with 5 molar equivalents of H₂O₂. Right: UV-vis absorbance spectra after addition of N-methylimidazole to 3+(CH₃CN)₄CuPF₆+5 molar equivalents of H₂O₂ in H₂O after 18 hours incubation.

FIG. 3 is Scheme 4 illustrating potential energy surface for the proposed reaction mechanism for the copper-catalyzed azide alkyne cycloaddition.

FIG. 4 is proposed catalytic cycle for the copper-catalyzed azide alkyne cycloaddition.

FIG. 5 is a-b) Effect of 6 and 7 on cell viability in TNBC cells treated with the complexes for 72 hours. Mean±s.e.m. n=3. c) Structure of AuDTC-alkyne used for click experiment.

### DETAILED DESCRIPTION

New compounds are provided having the chemical formula:

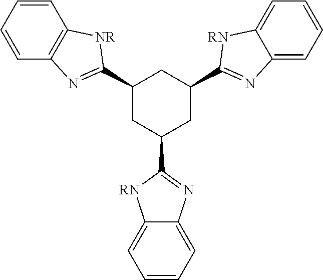

where R=—H, —C₄H₉ or —CH₂—CH₂—O—CH₂—CH₂—O—CH₃; or

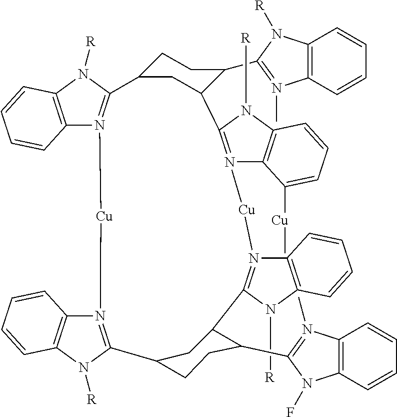

where R=—C₄H₉ or —CH₂—CH₂—O—CH₂—CH₂—O—CH₃.

Any of these compounds may be useful as reaction intermediates to produce CuAAC reaction catalysts. As described below, compounds with the chemical formula:

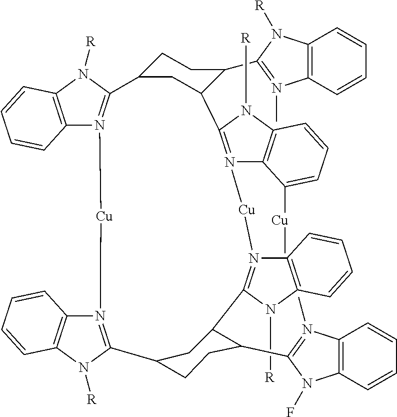

where R=—C₄H₉ or —CH₂—CH₂—O—CH₂—CH₂—O—CH₃ may be useful as CuAAC reaction catalysts.

### Ligand Synthesis

The synthesis of the cyclohexane tribenzimidazole ligands (Scheme 1) began with the condensation of cis,cis-1,3,5-cyclohexanetricarboxylic acid with o-phenylenediamine to form 1·3HCl in good yield.

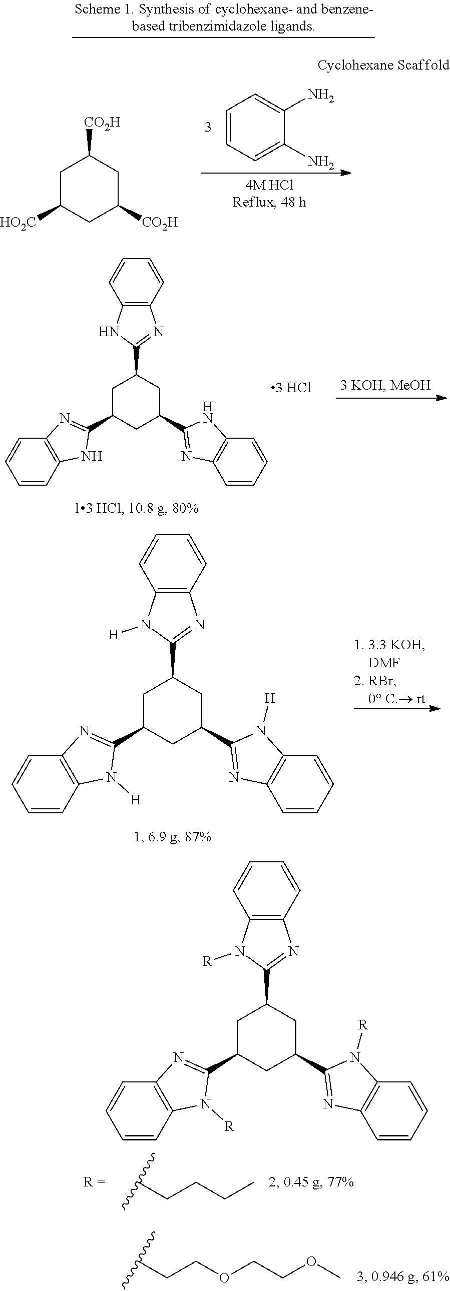

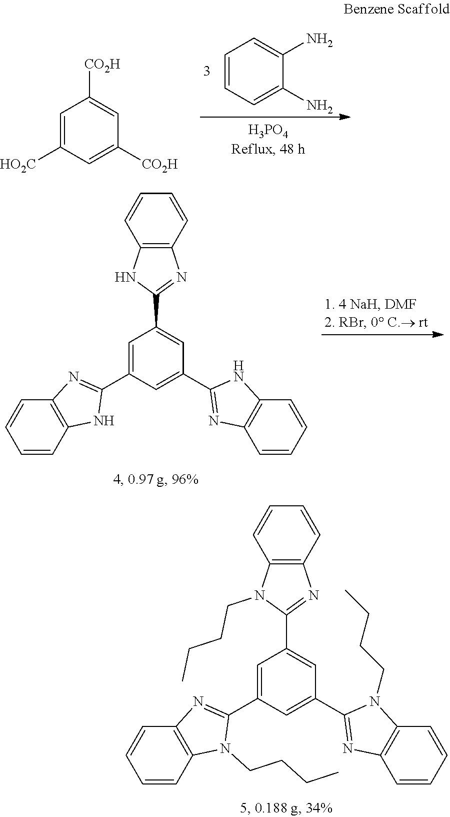

X-ray quality single crystals of 1·3HCl were grown from slow cooling of an NMR sample in F₂O. Neutralizing the hydrochloride salt with KOH smoothly provided 1 in 87% yield. X-ray quality single crystals of 1 were grown by slow cooling of a saturated solution of acetone. Deprotonation of 1 with KOH and reaction with butyl bromide or 1-bromo-2-(2-methoxyethoxy)ethane led to ligands 2 and 3 in good yield (77% for 2 and 61% for 3). The structure of 2 was obtained from X-ray quality single crystals grown from slow evaporation of a concentrated acetonitrile, CH₃CN, solution. Additionally, X-ray quality single crystals of 3 were obtained by diffusing diethyl ether, Et₂O, into a methanolic solution of 3. The structures of the ligands were resolved by X-ray crystallography.

Synthesis of the benzene tribenzimidazole ligand was achieved by following the same approach described above. Briefly, starting from 1,3,5-benzenetricarboxylic acid, condensation with o-phenylenediamine afforded 4. Alkylation with butyl bromide provided 5. While the non-alkylated versions of 1 and 3 exhibited only two shifts in the aromatic region, which corresponded to the two equivalent types of protons on the benzimidazole ring, the alkylated versions (2 and 5) had three resonances in the aromatic region. One of them was an overlapping multiplet corresponding to the two middle benzimidazole arene protons, and two resonances that split into a pair of one upfield and one downfield resonance, corresponding to the protons furthest and nearest the alkylated nitrogen, respectively.

### Metal Complex Synthesis

Once synthesized, each ligand was metalated with (CH₃CN)₄CuPF₆ (Scheme 2). Upon addition of Cu(I) to ligand 2, a marked shift in the ¹H NMR spectrum was observed after 1 hour at 60° C. in CH₃CN. While the aromatic region for 2 contained three apparent signals, the spectrum of the product 6 contained four signals, which indicated each of the four benzimidazole protons were in a different electronic environment. Another shift was observed in the resonances corresponding to the methylene protons a to the N atom, from ˜4.2 ppm to ˜4.5 ppm. We reasoned that a shift in this signal would result from bonding of the benzimidazole N atom. The cyclohexane axial proton bound to the unsubstituted carbon shifted downfield after reaction with Cu(I), from ˜3.45 ppm to ˜4.55 ppm. We reasoned that this proton could be shifted downfield if it were in closer proximity to the copper atom in the resulting complex. Absent from the spectrum after workup was a signal that corresponded to free or chelated CH₃CN (or other solvate). Each of the observed ¹H NMR resonance shifts indicated to us that the ligand was complexed to the Cu(I) metal center and that the coordination sphere likely did not involve a solvate.

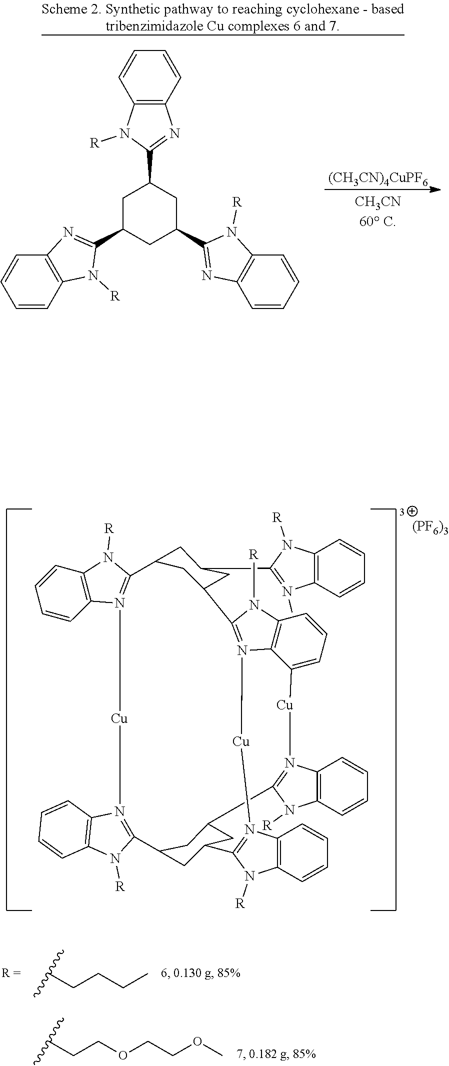

When 3 was used to chelate the copper precursor and form complex 7, we observed a similar pattern of resonances shifting in the ¹H NMR spectrum after 1 hour at 60° C. Both the α-amine carbon's protons and the cyclohexane's methylene protons shifted, and the aromatic signals exhibited the same pattern. Four distinct single proton aromatic resonances were present for each benzimidazole proton, as observed for 6.

Additionally, 5 was used to chelate the copper precursor and form complex 8. After 1 hour at 60° C. in CH₃CN, we observed the three aromatic signals change to five. The benzene singlet shifted upfield (from ˜8.4 ppm to ˜7.2 ppm), which we reasoned could be caused by increased shielding due to proximity to the alkyl substituents. The apparent triplet signal corresponding to six protons present in 5 split into two doublets corresponding to three protons, one upfield (˜7.4 ppm) and one downfield (˜8.1 ppm). The overlapping triplets corresponding to six protons at ˜7.25 ppm in 5 split into two resonances corresponding to three protons, one upfield (˜7.45 ppm) and downfield (˜7.75 ppm). With these data, we reasoned that the ligand was complexed to Cu(I) using each benzimidazole. After condensing the reaction mixture, however, the product signals further split into additional aromatic resonances, which indicated formation of additional side-products.

### Crystal Structures of Cu (I) Complexes

X-ray quality single crystals of 6 were grown by vapor diffusion crystallization using a solution of dichloromethane and diethyl ether antisolvent. Crystals of 7 were also grown by vapor diffusion, using a methanol solution and with diethyl ether antisolvent.

In both complexes, the benzimidazole substituents are in the equatorial position. Average cyclohexane C—C—C bond angles are 110.390 for 6 and 110.090 for 7, which indicated less overall strain in 7. In the structure of 6, the three copper centers form a triangle with sides of length 5.573, 5.573, and 5.264 Å long. In 7, the triangle is equilateral, with the copper atoms each 5.362 Å apart. In 6, two of the copper centers have Cu—N bond lengths of 1.862(5) and 1.865(5) Å, while the third has shorter Cu—N bond lengths of 1.750 Å. Each of the copper centers in 7 has the same Cu—N bond lengths of 1.861(3) and 1.856(3) Å. The two cyclohexyl rings in 7 are nearly vertically oriented, while in 6 there is a slight rotational offset between the two rings, likely due to the difference in Cu—N bond length observed in 6. Also of note, as we reasoned from the downfield shifted cyclohexane resonance near ˜4.55 ppm in the ¹H NMR spectra of 6 and 7, three axial protons are oriented towards the interior of the trinuclear dimer.

Single crystals of 16 were grown from vapor diffusion crystallization (outside vial=Et₂O, inside vial=CH₃CN). Available data indicated the presence of a tetranuclear tetramer rather than the trinuclear dimer seen for 6 and 7. Each copper ion was bound in a tetrahedral geometry to 3 benzimidazole N atoms and a central anion. Diffraction data was not of high enough quality for publication, however.

### Catalytic Trials

With the metal complexes in hand, we sought to test the catalytic activity in the CuAAC reaction (Scheme 3, Table 1). Typical reaction conditions for the CuAAC involve a slight excess of alkyne, a catalyst in low loading (down to <1% in some cases) a polar reaction solvent, and low temperature (down to room temperature). We monitored the reactions and quantified reaction products by gas chromatography (GC).

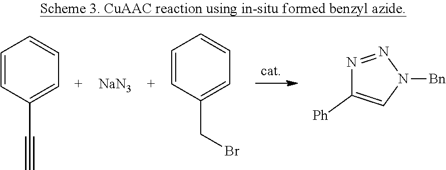

TABLE 1
Catalytic CuAAC reactions with catalyst 6.^{a}

| | | Catalyst | Time | Conversion |
|---|---|---|---|---|
| Entry | Catalyst | Loading (mol %) | (h) | (%) |
| 1^{b} | (CH₃CN)₄CuPF₆ | 1 | 24 | 22 |
| 2^{c} | 6 | 0.33 | 24 | 98 |
| 3^{d} | 6 | 0.5 | 4 | 100 |

^{a}Unless otherwise noted, reaction conditions are catalyst, phenylacetylene, benzyl bromide, and sodium azide dissolved in MeOH, allowed to react at 60° C. and at the time indicated product conversion was measured using GC.
^{b}phenylacetylene- 1.6 mmol, benzyl bromide- 1.8 mmol.
^{c}phenylacetylene- 0.55 mmol, benzyl bromide- 0.60 mmol.
^{d}reaction temperature: room temperature.

When no ligand was used (Table 1 Entry 1, 1 mol % (CH₃CN)₄CuPF₆ only) the reaction proceeded very slowly (31% after 24 hour at room temperature and 22% after 24 hour at 60° C.). (CH₃CN)₄CuPF₆ has been previously shown to be ineffective for the click reaction due to the strong donating nature of the acetonitrile ligands. When complex 6 was used as the catalyst in 0.33 mol % (1 mol % Cu⁺) loading in MeOH, 98% conversion to triazole was observed after 24 hours at both room temperature and 60° C. (Table 1 Entry 2). When 0.5 mol % loading of 6 was used, conversion to triazole was nearly quantitative at 4 hours in MeOH at room temperature (Table 1 Entry 3). To check whether it was necessary to use an isolated sample of complex 6, an in-situ formed 6 (2+(CH₃CN)₄CuPF₆) was used, and it was found to have nearly identical conversion to triazole after 4 hours (Table 2, Entry 1 vs. Entry 2).

TABLE 2
Catalytic CuAAC reactions with catalyst 6.^{a}

| | | Catalyst | | Time | Conversion |
|---|---|---|---|---|---|
| Entry | Catalyst | Concentration | | (hr) | (%) |
| 1 | 6 | 20 | mM | 4 | 98 |
| 2 | 2 + (CH₃CN)₄CuPF₆ | 20 | mM | 4 | 98 |
| 3 | 3 + (CH₃CN)₄CuPF₆ | 20 | mM | 4 | 97 |
| 4 | 1 + (CH₃CN)₄CuPF₆ | 20 | mM | 4 | 18 |
| 5 | 4 + (CH₃CN)₄CuPF₆ | 20 | mM | 4 | 25 |
| 6 | 5 + (CH₃CN)₄CuPF₆ | 20 | mM | 4 | 16 |
| 7^{b} | 3 + (CH₃CN)₄CuPF₆ | 0.5 | μM | 18 | 30 |
| 6^{b} | 3 + (CH₃CN)₄CuPF₆ | 1.0 | μM | 18 | 60 |
| 9^{b} | 3 + (CH₃CN)₄CuPF₆ | 5 | μM | 18 | 90 |
| 10^{b} | 3 + (CH₃CN)₄CuPF₆ | 10 | μM | 18 | 98 |

^{a}Unless otherwise noted, reaction conditions are catalyst, phenylacetylene (1.0 μmol), benzyl azide (1.1 μmol) dissolved in water allowed to react at room temperature and at the time indicated product conversion was measured using GC. Each entry is an average of two runs.
^{b}reaction temperature: 35° C.

Because it was not necessary to isolate the trinuclear dimer complex, we next screened ligands 1, 3, 4, 5. Ligands 1 and 4 do not have alkyl substituents and thus are less strong donors for the Cu(I) center. Ligand 3 has 2-(methoxyethoxy)ethane substituents instead of butyl to improve solubility in aqueous environments and 5 has a butyl substituent. 4 and 5 both have benzene rings instead of the cyclohexyl ring, which should change the overall complex electronic and steric profiles. When each of the ligands was complexed to Cu(I) in-situ and used as a catalyst, the glycolated ligand 3 exhibited nearly quantitative conversion to triazole after 4 hours at room temperature, as was the case for the butylated dimer 2. Non-alkylated 1, and the two benzene derivatives 4 and 5 all gave similar conversions and were in general much less active (˜25% for 5, ˜18% for 1, and ˜16% for 4). These data make it clear that the cyclohexyl ring was necessary, and that alkylation was important for maximum catalytic activity.

Since biological systems are the ultimate target for our CuAAC catalysts, we tested catalytic activity of in-situ formed 7 in water. Using 1 mM scale in reference to azide, we varied the catalyst concentration from 50 μM down to 0.5 μM at room temperature, or at the biologically relevant 35° C. After 18 hours of shaking or stirring the reactions, it was found that at ambient conditions there was ˜36% conversion at a catalyst concentration of 50 μM, ˜13% at 10 μM and 5 μM, 8% at 1 μM, and 6% at 0.5 μM. At 35° C., the 50 mM loading indicated 75% conversion after 1 hour. There was quantitative conversion after 18 hours when the catalyst concentration was 10 μM, ˜88% at 5 μM, 64% at 1 μM, and 33% at 0.5 μM.

To investigate substituent effects on the rate of reaction, NMR-scale catalytic experiments were performed. Catalytic tests included four terminal alkynes: phenylacetylene, 1-hexyne, 1-ethynylanisole and 1-ethynyl-4-nitrobenzene. The resulting data shows the nitrobenzene-substituted alkyne was fastest, while the 1-hexyne was the slowest. The electron rich methoxy-substituted aryl alkyne reacted at a similar rate to the unsubstituted phenylacetylene.

To demonstrate the dependence of the rate on the catalyst loading, the concentration of the complex was tested at 1, 2 and 4 mM. The reaction rate showed a linear dependence on the catalyst concentration.

### Stability of the Complex

One of the most important aspects of catalysts for the biologically compatible CuAAC reaction is the ability to perform the reaction while not forming any ROS. Most Cu (I) complexes can be rapidly oxidized by oxygen to Cu (II) and form O₂—, one of the first ROS that can then go on to react in myriad ways. Complexes 6 and 7 did not change color in the presence of air or moisture over the course of weeks, either in solid or solution. To further verify this, we performed EPR measurements on solutions of 6 in the absence and presence of H₂O₂. Before adding oxidant, we did not observe any EPR active species and after H₂O₂ addition, we again observed no EPR active species. Because Cu²⁺ is EPR active, this indicated that no Cu²⁺ was formed due to oxidation of Cu (I) metal centers present in 6.

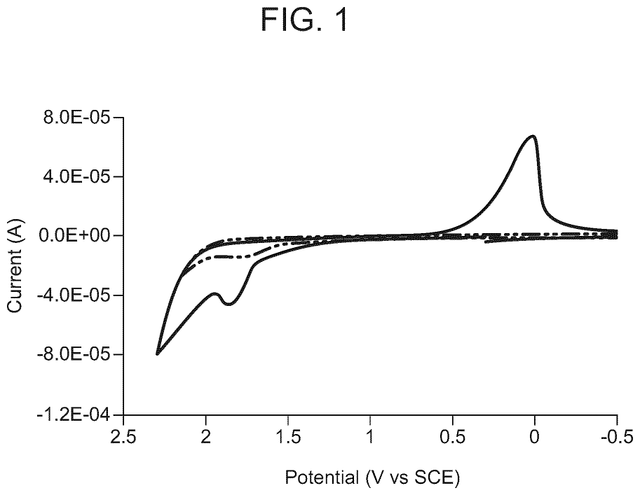

The oxidation potential of complex 7 was measured using cyclic voltammetry (CV) in organic and aqueous solvents (FIG. 1). We first dissolved 7 in CH₂Cl₂, used a glassy carbon working electrode, Pt counter electrode, and Pt pseudo-reference electrode, and scanned positively from 0 V. An oxidation wave was observed at 1.85 V vs SCE after conversion from the Fc⁺/Fc couple using the equation V vs. SCE=V vs. Fc⁺/Fc-0.46 V. An irreversible reduction wave at ˜0 V vs. SCE was also observed that was ascribed to a Cu⁺/Cu⁰ reduction. We then dissolved 7 in 0.1 M LiClO₄ in H₂O and measured the CV with a glassy carbon working electrode, Pt counter electrode, and Hg/Hg₂SO₄ reference electrode. We did not observe an oxidation wave in H₂O before reaching the end of the functional solvent window.

To determine the identity of the oxidation event at 1.85 V vs SCE, we then subjected the uncoordinated ligand 3 to CV and observed an irreversible oxidation near the same potential as we observed for 7. This led us to conclude that both peaks represent the oxidation of the ligand, rather than the Cu (I) centers.

This oxidation potential observed is much higher than found for (CH₃CN)₄CuPF₆ (0.207 V vs Ag/AgCl in CH₃CN), tripodal pyridyl Cu complexes (˜0.30 V vs Ag/AgCl in H₂O), and tripodal amine Cu (I) benzimidazole complexes (0.187 V vs. Ag/AgCl in MeCN). It has been shown by others that linear Cu (I) complexes are much more resistant to oxidation than are Cu (I) complexes of higher coordination number, which typically oxidize to Cu (II) very quickly. Therefore, we reason that the much higher stability we observe for this complex is due to the linear coordination geometry observed in 6 and 7.

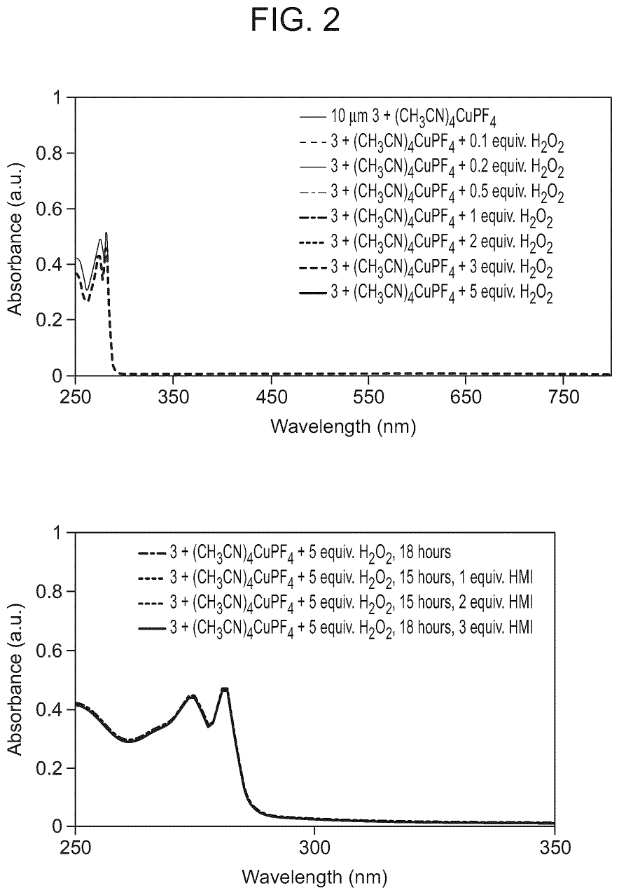

We then tested the chemical oxidation of solutions of 7 by UV-visible spectroscopy in CH₃CN. Increasing amounts of H₂O₂ were added and the absorption spectrum was measured (FIG. 2).

Without the addition of oxidant, an absorption peak at 280 nm is the lowest energy feature in the spectrum. Aqueous solutions of Cu²⁺ ions absorb visible light, typically due to low energy d-d transitions. Therefore, if Cu²⁺ was formed, we would expect to observe the appearance of a new absorption in the visible region. It was observed that upon addition of up to five molar equivalents of peroxide, there was no appreciable change in the absorption spectrum after 18 hours at room temperature. With the data that the complex did not oxidize upon contact with H₂O₂, we then also added N-methylimidazole to the cuvette with in-situ formed 7 and five equivalents of H₂O₂ and found again no change in the absorption spectrum. This experiment indicated that 7 is stable and did not undergo and changes in the presence of N-methylimidazole, peroxide, or in mixtures of the two.

Proposed Mechanism and Catalytic Cycle from Computational Results

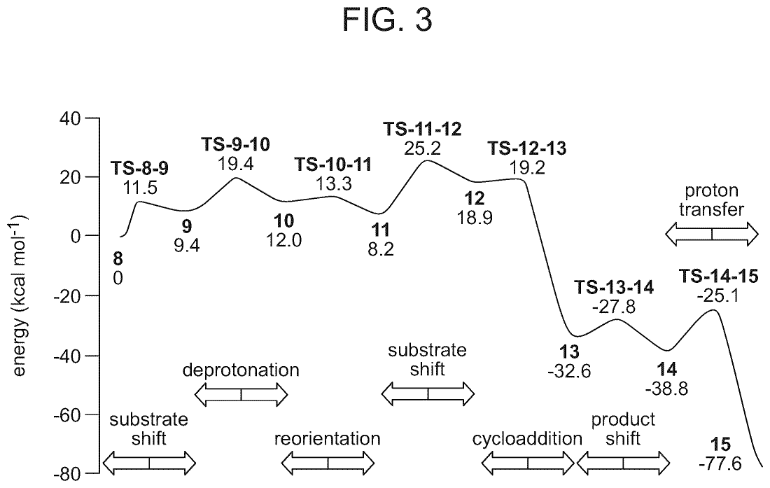

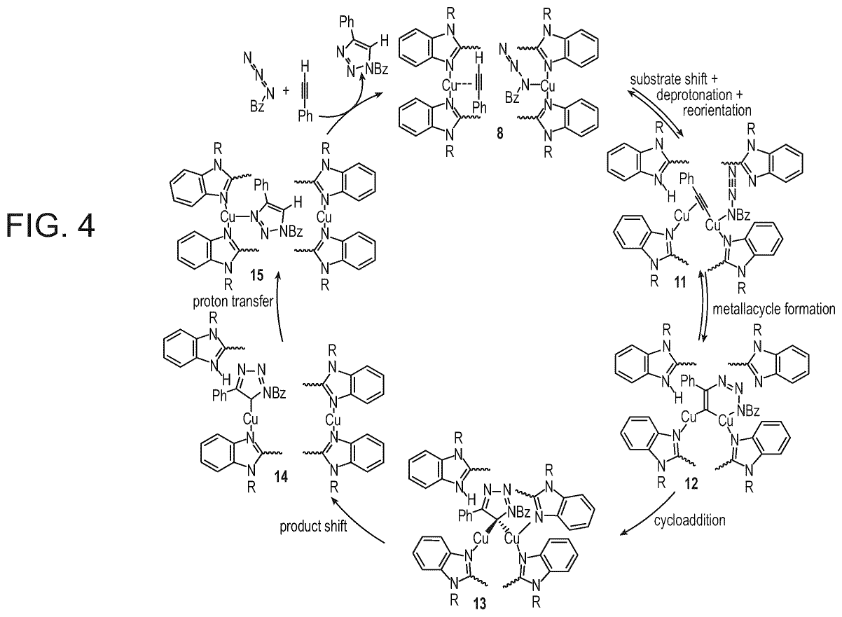

The proposed mechanism for the copper-catalyzed azide alkyne cycloaddition for the trinuclear copper complexes (6 and 7) is given in Scheme 4 (See FIG. 3) and the catalytic cycle in FIG. 4. The results suggest a stepwise mechanism involving metallacycle intermediates and the requirement of two equivalents of copper in the turn-over limiting step in the catalytic cycle. This proposed mechanism is consistent with previously reported mechanistic studies on model systems. Utilizing complex 6, a slight simplification was made: the R groups were modeled as methyl groups to generate complex 6^{Me}. Coordinating alkyne and organic azide to complex 6^{Me} affords complex 8. Complex 8 is the structure with an η²-alkyne ligand coordinated to one copper center and the κ¹-nitrogen organic azide coordinated to a second copper center.

The alkyne must shift (through TS-8-9) in order to come to structure 9 that will then transfer the proton (through TS-9-10) from the coordinated alkyne to the nitrogen of a benzimidazole ligand to form structure 10 with a κ¹-coordinated deprotonated alkynyl ligand to the same copper center. From there, this κ¹-coordinated alkynyl ligand must reorient (through TS-10-11) to form a bridging η² coordination to the same copper center, while simultaneously forming a new κ¹-coordination to the second copper center that already has the κ¹-coordinated organic azide (structure 11). From structure 11, TS-11-12 forms a heterometallacyclohexenyl species with one of the carbon centers in the cupracycle κ¹-coordinated to another copper center to form structure 12.

From structure 12, TS-12-13 finalizes the cycloaddition by ring-size reduction to form structure 13 with the μ²-C-five-membered triazolyl anion coordinated simultaneously to the two copper centers. Structure 14 is formed (through TS-13-14) after the shift of the triazolyl anion to become terminally κ¹-coordinated to a single copper center. Finally, the proton transfers from the protonated benzimidazolium ligand (through TS-14-15) to form the κ¹-N-copper-coordinated triazole product (15), which can dissociate from the catalyst to complete the catalytic cycle. The proposed mechanism is consistent with previously reported proposals.

### Intracellular Cu(I)-Catalyzed CuAAC

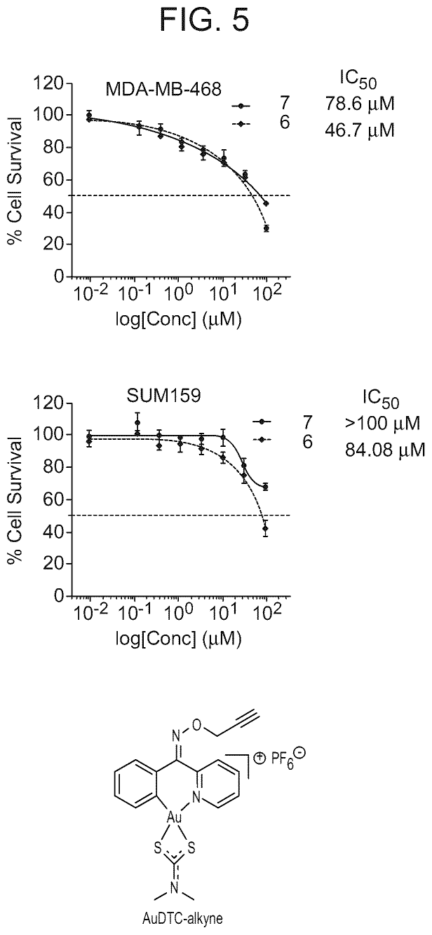

Following the outcome of our catalytic trials, we rationalized the Cu(I) complexes under examination will demonstrate applicability for intracellular click chemistry, whereas avoiding the use of THPTA and ascorbate. We first evaluated the cytotoxicity of the complexes (6 and 7) in triple-negative breast cancer cells, MDA-MB-468 and SUM-159 using MTT assay. 6 showed cytotoxicity with IC₅₀ values of 46.7 μM and 84.08 μM in MDA-MB-468 and SUM-159 cell lines respectively (FIG. 5a-b). 7 was less toxic, with IC₅₀ values of 78.6 μM and >100 μM in MDA-MB-468 and SUM-159 cell lines respectively (FIG. 5a-b).

These IC₅₀ values for 6 and 7 are more than twice and three times larger, respectively, than the IC₅₀ value in MDA-MB-468 cells observed for the most commonly employed CuAAC catalyst, Cu tris-(hydroxypropyltriazolyl)-methylamine (THPTA), of 22.4 μM. We selected 7 for the subsequent in vitro click chemistry study. 7 effectively catalyzed the click reaction between a mitochondria-targeting gold-based complex bearing an alkyne handle (AuDTC-alkyne) and azide fluorescein. Colocalization of AuDTC-alkyne with the mitochondria-specific dye MitoTracker Red CM-H₂XRos (MTR) showed mitochondria localization with a Pearson correlation of 0.7. For comparison, we performed the traditional CuSO₄ CuAAC reaction using its standard reaction condition and found that the reaction occurs in cells with the alkyne substrate as expected.

The information presented in this document may be summarized as follows:
A. List of Complexes
   a. BTBIC CuPF₆ Complex: (Cis,cis-1,3,5-cyclohexanetribenzimidazole-1-butyl-2-yl)₂ (Copper(I) hexafluorophosphate)₃, C₇₈H₉₆N₂₂Cu₃P₃F₁₈
   b. TBIC-PEG2 CuPF₆ Complex: (Cis,cis-1,3,5-cyclohexanetribenzimidazole-1-(2-(2-methoxyethoxy)ethyl)-2-yl)₂ (Copper(I) hexafluorophosphate)₃, C₈₄H₁₀₈O₁₂N₁₂Cu₃P₃F₁₈
B. Steps to synthesize complexes:
   a. Condensation of o-phenylenediamine with cis,cis-1,3,5-cyclohexanetricarboxylic acid in 4M HCl

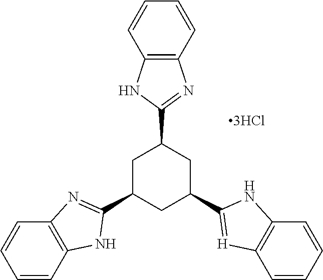

Cis,cis-1,3,5-cyclohexanetribenzimidazole-2-yl·3HCl (1·3HCl): 5.00 grams (23.1 mmol, 1 mol. equiv.) of cis,cis-1,3,5-cyclohexanetricarboxylic acid was added to a 500 mL RBF and dissolved in 92.5 mL of 4 M HCl. 15.0 grams (139 mmol, 6 mol. equiv.) of o-phenylenediamine was added to the flask. The reaction was heated to 130° C. to stir under reflux for 2 days. The reaction was cooled to room temperature and the product precipitated by addition of acetone. The product was filtered via vacuum filtration and successively washed with another acetone, ethyl acetate, and diethyl ether, and left to dry under vacuum. 10.8 g claimed from the frit for an 80% yield.

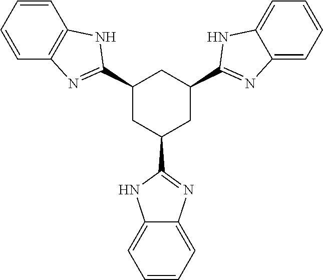 [SMILES: c1ccc2[nH]c([C@H]3C[C@@H](c4nc5ccccc5[nH]4)C[C@@H](c4nc5ccccc5[nH]4)C3)nc2c1]

Cis,cis-1,3,5-cyclohexanetribenzimidazole-2-yl (1): 10 grams (18 mmol, 1 mol. equiv.) of 1.3HCl was mixed into 26 mL methanol. 3.1 grams (55 mmol, 3 mol. equiv.) of KOH was added and the flask was stirred until the salt crashed out. The solid was filtered off and lightly washed with cold methanol. The filtrate was rotovapped down for 6.9 grams of product, 87% yield.
b. S_{N}2 Substitution with a butyl- or PEG₂-bromide to provide the respective ligand

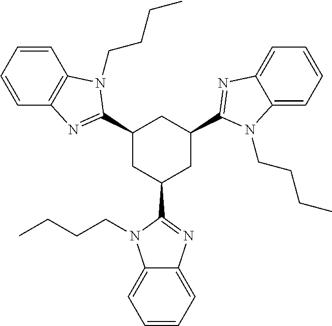 [SMILES: CCCCn1c2ccccc2nc1[C@H]1C[C@@H](c2nc3ccccc3n2CCCC)C[C@@H](c2nc3ccccc3n2CCCC)C1]

Cis,cis-1,3,5-cyclohexanetribenzimidazole-1-butyl-2-yl (2): To a flame-dried 250 mL RBF charged with a stir bar, 462 mg (0.967 mmol, 1 mol. equiv.) of 1 was added. 9.7 mL of dry DMF was added via syringe and the solution was cooled to 0° C. 514 mg (3.19 mmol, 3.3 mol. equiv.) of KOH was added and the reaction was stirred for 15 minutes before adding 0.34 mL (3.19 mmol, 3.3 mol. equiv.) of 1-bromobutane. The reaction formed a cloudy brown mixture and was allowed to warm to room temperature and left to stir overnight. The reaction was quenched with water, and the product was extracted 3× with DCM. The organic layer was combined and washed 4× with water, dried over sodium sulfate, and then rotovapped to dryness. 450 mg of 2 were claimed for a 77% yield.

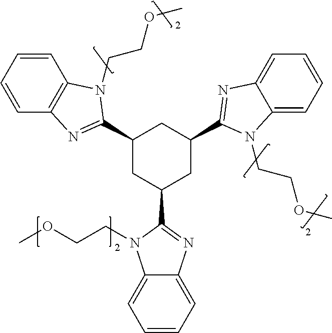

Cis,cis-1,3,5-cyclohexanetribenzimidazole-1-(2-(2-methoxyethoxy)ethyl)-2-yl (3): 1.00 grams (2.09 mmol, 1 mol. equiv.) 1 was deposited in a 50 mL RBF charged with a stir bar and dissolved in 21 mL DMF. The solution was cooled to 0° C. and 0.388 grams (6.91 mmol, 3.3 mol. equiv.) KOH was added to the solution. The solution was allowed to stir for 15 minutes and 2.80 mL (18.8 mmol, 9 mol. equiv.) 1-bromo-2-(2-methoxyethoxy)ethane was added. The reaction was left to stir overnight and warm to room temperature. Once the reaction reached completion, the reaction was condensed under reduced pressure by forming a toluene/DMF azeotrope. The product was purified via flash chromatography using a 5% MeOH/DCM solvent system. 0.946 grams of 3 were collected for a 61% yield.
c. Complexation of tetrakis(acetonitrile) copper hexafluorophosphate in acetonitrile solvent

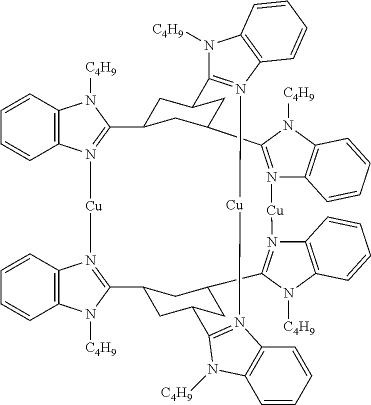

(Cis,cis-1,3,5-cyclohexanetribenzimidazole-1-butyl-2-yl)₂(Cu(I)PF₆)₃ (6): To a 2-dram vial charged with a stir flea, 100 mg (0.166 mmol, 1 mol. equiv.) 2 was added, followed by 3.3 mL acetonitrile. 93 mg (0.250 mmol, 1.5 mol. equiv.) tetrakis(acetonitrile) copper(I) hexafluorophosphate was added, dissolving the mixture, and the reaction was left to stir overnight at 60 C. Reaction cooled to room temperature and rotovapped to a crystalline white powder. 130 mg collected for an 85% yield.

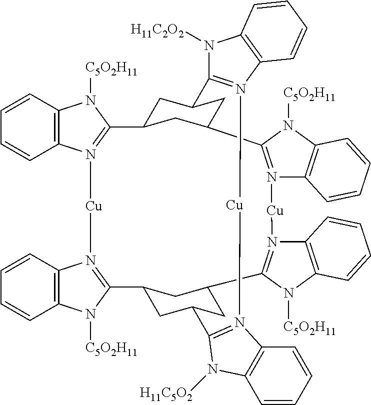

(Cis,cis-1,3,5-cyclohexanetribenzimidazole-1-(2-(2-methoxyethoxy)ethyl)-2-yl)₂(Cu(I)PF₆)₃(7): To a 3-dram vial charged with a stir bar, 0.150 grams (0.203 mmol, 1 mol. equiv.) 3 was added followed by 4.06 mL acetonitrile. 0.113 grams (0.304 mmol, 1.5 mol. equiv.) tetrakis(acetonitrile) copper(I) hexafluorophosphate was added to the vial, dissolving the mixture, and the reaction was stirred overnight at 60° C. The reaction was monitored by NMR. Upon completion, the solvent was removed under reduced pressure. 0.182 grams collected for an 85% yield.
C. Experimental example of how to use the catalyst
   a. Typical cycloaddition between benzyl azide and phenylacetylene
      i. Conditions: 1 mL scale in water. 1 μmol azide, 1.1 μmol alkyne. 50 PPM TBIC-PEG₂ CuPF₆ catalyst formed in-situ. Performed at 35° C. for 18 hours in air.
         1. 995.4 μL water was added to a 3-dram vial charged with a stir flea.
         2. 1 μL of a 1 mM stock solution of TBIC-PEG₂ (3) in DMSO was added to the vial, followed by 1.5 μL of 1 mM stock solution of Cu(I)(MeCN)₄PF₆ in acetonitrile. This forms the TBIC-PEG2 CuPF₆ complex (7) in-situ.
         3. 1 μL of a 0.1 M benzyl azide stock solution in DMSO was added to the vial.
         4. At the start time, 1.1 μL of a 0.1 M phenylacetylene stock solution in DMSO was added to the reaction, initiating catalysis.
         5. The vial was then left to stir at 35° C. for 18 hours.
         6. At the 18 hour mark, the reaction is stopped by addition of 0.5 mL THF.
         7. The yield quantified by injecting an aliquot of the reaction on a gas chromatograph and calculating the amount of product formed with a calibration curve.
   b. Using the catalyst in vivo
      i. Conditions: Performed using live cells. Uses 100 μM preformed TBIC-PEG₂ CuPF₆ catalyst.
         1. MDA-MB-468 cells were seeded at 3×105 cells/mL on 35 mm glass-bottom petri dishes and incubated overnight in appropriate growth medium (DMEM) at 37° C.
         2. Cells were allowed to adhere overnight after which media was removed and cells were washed with PBS twice before treatment with AuDTC-alkyne (50 μM) for 1 hour. The control dishes were left untreated.
         3. After treatment with test compounds, cells were washed three times with PBS followed by treatment with mitotracker red (200 nM) for 35 min
         4. The cells were washed three times and fixed with 4% PFA at 37° C. for 15 min.
         5. Cells were washed three times after fixing and permeabilized with 0.2% triton-X for 1 hour at room temperature.
         6. Cells were washed three times with PBS after permeabilization.
         7. Azide fluorescein (final concentration of 100 μM) in PBS, was mixed with TBIC-PEG2 CuPF₆ complex (100 μM) for a total volume of 3 mL in PBS in a 15 mL tube.
         8. The mixture was added to the cells for 1 hour and the cells were kept at 4° C. After 1 hour, cells were washed three times, and the Hoechst dye (1 μg/mL) was added for 15 min to stain the nuclei.
         9. Cells were washed with PBS and mounted on the Nikon A1R confocal microscope with 60× oil objective. Images were analyzed with NIS viewer.
D. The air-stable preformed linear Cu(I) complexes 6 and 7 may be used to catalyze the CuAAC reaction, a standard technique in the biochemist's toolkit. The complexes are synthesized in only three steps from readily available starting materials, and in good yield. While other air-stable linear Cu(I) complexes bearing N-donating ligands have been reported in the past, this marks their first effective use in the CuAAC reaction. To catalyze the reaction, a nontoxic complex already in the catalytically active Cu(I) state is used, rather than the current standard of a Cu(II) salt, reducing agent, and an excess of ligand. In this way, the need for the reducing agent, sodium ascorbate, to conduct the reaction is eliminated. In doing so, the amount of reactive oxygen species—which can cause cellular apoptosis—that are produced by the sodium ascorbate in Cu(II)-catalyzed cycloadditions is advantageously minimized. In turn, this also reduces the amount of ligand that is necessary to perform the reaction. Thus, catalyst 6 and 7 improve upon the current standard in two ways; by minimizing the risk of reactive oxygen species, and reducing the cost of running the reaction by eliminating the need for sodium ascorbate and, therefore, excess ligand.

Each of the following terms written in singular grammatical form: “a”, “an”, and “the”, as used herein, means “at least one”, or “one or more”. Use of the phrase “One or more” herein does not alter this intended meaning of “a”, “an”, or “the”. Accordingly, the terms “a”, “an”, and “the”, as used herein, may also refer to, and encompass, a plurality of the stated entity or object, unless otherwise specifically defined or stated herein, or, unless the context clearly dictates otherwise. For example, the phrase: “a thermosetting resin”, as used herein, may also refer to, and encompass, a plurality of thermosetting resins.

Each of the following terms: “includes”, “including”, “has”, “having”, “comprises”, and “comprising”, and, their linguistic/grammatical variants, derivatives, or/and conjugates, as used herein, means “including, but not limited to”, and is to be taken as specifying the stated component(s), feature(s), characteristic(s), parameter(s), integer(s), or step(s), and does not preclude addition of one or more additional component(s), feature(s), characteristic(s), parameter(s), integer(s), step(s), or groups thereof.

The phrase “consisting of”, as used herein, is closed-ended and excludes any element, step, or ingredient not specifically mentioned. The phrase “consisting essentially of”, as used herein, is a semi-closed term indicating that an item is limited to the components specified and those that do not materially affect the basic and novel characteristic(s) of what is specified.

Terms of approximation, such as the terms about, substantially, approximately, etc., as used herein, refers to ±10% of the stated numerical value.

While the novel compounds and the methods for the production of the compounds and the uses of the compounds have been illustratively described and presented by way of specific exemplary embodiments, and examples thereof, it is evident that many alternatives, modifications, or/and variations, thereof, will be apparent to those skilled in the art. Accordingly, it is intended that all such alternatives, modifications, or/and variations, fall within the spirit of, and are encompassed by, the broad scope of the appended claims.

What is claimed:

1. A compound having the chemical formula 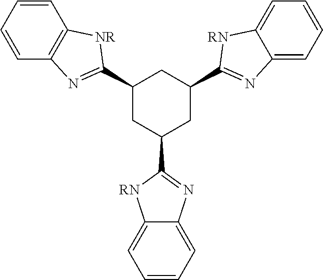
where R=—H. —C₄H₉ or —CH₂—CH₂—O—CH₂—CH₂—O—CH₃.

2. A compound having the chemical formula: 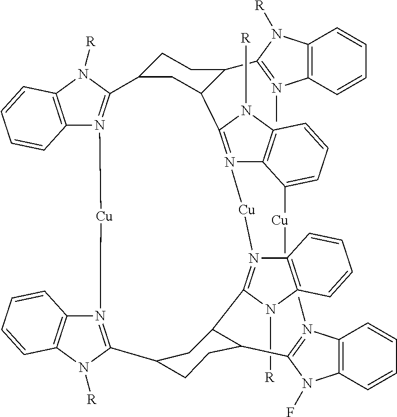
where R=—C₄H₉ or —CH₂—CH₂—O—CH₂—CH₂—O—CH₃.

3. A method of producing a CuAAC reaction catalyst, comprising:
using as a reaction intermediate, a compound selected from a group consisting of; 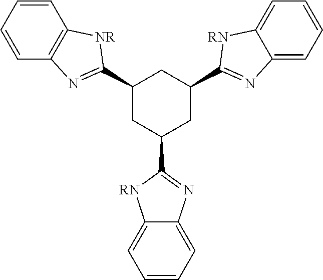
where R=—H. —C₄H₉ or —CH₂—CH₂—O—CH₂—CH₂—O—CH₃; or 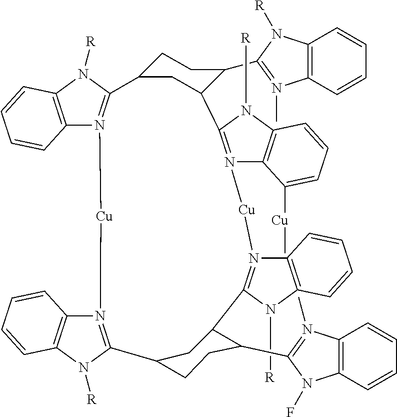
where R=—C₄H₉ or —CH₂—CH₂—O—CH₂—CH₂—O—CH₃.

4. A method of producing a CuAAC reaction catalyst, comprising:
reacting a cis-cis-1,3,5-cyclohexanetribenzimidazole-2-yl-based compound with tetrakis(acetonitrile) copper(I) hexafluorophosphate to form the CuACC reaction catalyst.

5. The method of claim 4 wherein the CuACC reaction catalyst has a chemical formula: 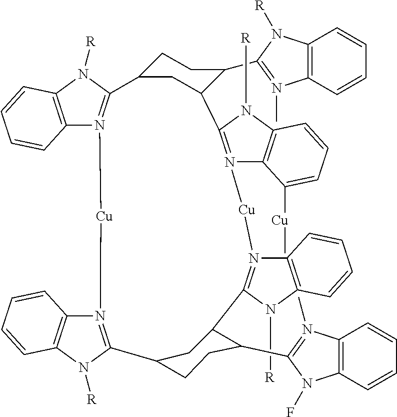
where R=—C₄H₉ or —CH₂—CH₂—O—CH₂—CH₂—O—CH₃.

6. A method for preparing a cycloaddition compound from a reaction catalyzed by a heterogeneous copper catalyst, comprising:
mixing an alkyne component and a azide component in an appropriate solvent to form a solution; and
contacting the solution with a CuAAC reaction copper(I) catalyst derived from a cis-cis-1,3,5-cyclohexanetribenzimidazole-2-yl-based intermediate compound wherein, upon contact with the CuAAC reaction copper(I) catalyst, the alkyne component and the azide component react to form the cycloaddition compound.

7. The method of claim 6, including completing the contacting in an absence of a reducing agent.

8. The method of claim 6, including completing the contacting in an absence of sodium ascorbate.

9. The method of claim 6, wherein the CuAAC reaction copper(I) catalyst is a compound having a chemical formula: 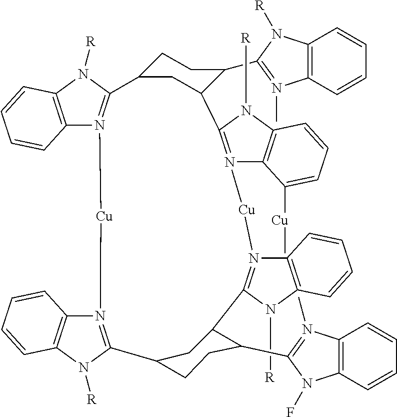
where R=—C₄H₉ or —CH₂—CH₂—O—CH₂—CH₂—O—CH₃.

10. The method of claim 9, including completing the contacting in an absence of a reducing agent.

11. The method of claim 9, including completing the contacting in an absence of sodium ascorbate.
